(19) United States
(12) Patent Application Publication
(10) Pub. No.: US 2004/0260319 A1
(43) Pub. Date: Dec. 23, 2004

(54) Device for generating an artificial constriction in the gastrointestinal tract

(76) Inventor: Walter Egle, Koblach (AT)

Correspondence Address: WENDEROTH, LIND & PONACK, L.L.P., 2033 K STREET N. W., SUITE 800, WASHINGTON, DC, 20006-1021, US

(21) Appl. No.: 10/857,925

(22) Filed: Jun. 2, 2004

(30) Foreign Application Priority Data
Jun. 4, 2003 (AT) A 861/2003

(51) Int. Cl. A61B 17/00; A61F 2/04

(52) U.S. Cl. 606/157; 128/899; 623/23.67; 604/909

(57) ABSTRACT

A device for generating an artificial constriction in the gastrointestinal tract comprises a band (1), which can be placed annularly about a particular portion of the gastrointestinal tract, and a closure device (2) for connecting the end regions of the band (1) placed annularly about a portion of the gastrointestinal tract, the band in the closed state of the closure device (2) encompassing a throughlet opening (6), the band (1) including a hollow chamber (13) fillable with a filling medium and extending at least over a large portion of the length of the band (1) and delimited on its side, facing the throughlet opening (6), by a diaphragm (11), by means of which the size of the throughlet opening (6) can be decreased by filling the hollow chamber (13). The band comprises further a second hollow chamber (14) separated from the first hollow chamber (13), the second hollow chamber also being fillable with a filling medium and on its side, facing the throughlet opening (6), delimited by a further diaphragm (12), by means of which the size of the throughlet opening (6) can be reduced by filling the second hollow chamber (14).

### BACKGROUND OF THE INVENTION

a) Field of the invention

The invention relates to a device for generating an artificial constriction in the gastrointestinal tract with a band, which can be placed annularly about a particular portion of the gastrointestinal tract, and a closure device for connecting the end regions of the band placed annularly about the portion of the gastrointestinal tract, which, in the closed state of the closure device, encompasses a throughlet opening, the band having a hollow chamber fillable with a filling medium and extending at least over a large portion of the length of the band and on its side, facing the throughlet opening, is delimited by a diaphragm by means of which the size of the throughlet opening can be reduced by filling the hollow chamber.

b) Description of related prior art

Such a device is disclosed in EP 0 702 529 B1, for example in the form of a gastric band. The device comprises a band placeable about the gastric inlet and is developed with a longitudinally extending interior hollow chamber. For its closing the band, placed annularly about the gastric inlet, comprises a closure device with a first closure part, disposed on one end of the band and comprising an insertion opening, and a second closure part, disposed at the other end of the band, which can be introduced through the insertion opening and latched relative to this opening. To constrict the passageway cross section of the throughlet opening of the band, and consequently of the gastric inlet, the hollow chamber of the band is filled with a filling medium and the quantity of the filling medium depends on the desired cross section of the passageway. For filling the band with the filling medium, an injection port connected with the band is provided. The port is implanted under the skin of the patient.

Apart from the development as a gastric band, a device of this type can in particular also be developed as an anal band for the closure of an, optionally, artificial anus.

One problem encountered in such devices comprises that these, sooner or later in the course of their use, can leak, such that their function is no longer ensured. Consequently, surgical removal and replacement of this band is required, which exposes the patient to corresponding strain. Such leaks occur in practice especially in the diaphragm delimiting the band toward the throughlet opening. Such leaks can occur for example due to material fatigue in the course of use or due to overfilling of the band. For so-called “early” leaks, which occur up to approximately one year after the placement of the band, such injuries are most often responsible, that have occurred through a surgical instrument during the surgical, in particular laparoscopic, placement of the band.

Further, WO 03/020183 A1 discloses a gastric band, which includes a band segment with which the opening of the stomach can be adjusted, in particular by means of a motor-driven setting unit drawing together the band segment. The band segment is encompasses by a viscoelastic material to protect the wall of the stomach, the tube, including the viscoelastic material, having in one embodiment example partitioning walls, which divide the tube into several chambers filled with the viscoelastic material.

### AIM AND SUMMARY OF THE INVENTION

One important aim of the invention is providing an improved device of the type described in the introduction, in which the frequency of surgical interventions necessitated by a leak of the band, is decreased.

This is attained according to the invention by a device for generating an artificial constriction in the gastrointestinal tract with

a band that can be placed annularly about a particular portion of the gastrointestinal tract, and

a closure device for connecting the end regions of the band placed annularly about the portion of the gastrointestinal tract, which, in the closed state of the closure device, encompasses a throughlet opening,

the band comprising a first hollow chamber fillable with a filling medium and extending at least over a large portion of the length of the band and delimited on its side, facing the throughlet opening, by a diaphragm, by means of which, through the filling of the hollow chamber, the throughlet opening can be decreased in size, and

the band furthermore comprising a second hollow chamber, separated from this first hollow chamber, fillable with a filling medium and extending at least over a large portion of the length of the band, and which on its side, facing the throughlet opening, is delimited by a further diaphragm, by means of which, through the filling of the second hollow chamber, the throughlet opening can be decreased in size.

In the event of damage to the diaphragm delimiting the throughlet opening and the escape of filling medium from the first hollow chamber, in a band according to the invention the second hollow chamber can be utilized for filling with the filling medium. Therefore, replacement of the band is also not required in the event of a leak in the diaphragm sealing the first hollow chamber against the throughlet opening. Overall, the average length of use of a band before the function assigned to it can no longer be fulfilled, is significantly extended.

In the annularly closed state of the band, the second hollow chamber favorably encompasses the first hollow chamber, the diaphragm delimiting the second hollow chamber on its side, facing the throughlet opening, advantageously delimiting the first hollow chamber on its side facing away from the throughlet opening.

In the case of a device according to the invention it can be provided that two injection ports or pumping devices are implanted in the patient, each of which is connected with a connection tubule, whose inner channels communicate, on the one hand, with the first hollow chamber and, on the other hand, with the second hollow chamber. If the first hollow chamber develops a leak, filling medium can be introduced into the second hollow chamber by means of the injection port or the pumping device, which is connected with the connection tubule communicating with the second hollow chamber. It is also conceivable and possible that only one injection port or pumping device is implanted in the patient, and thereon initially the connection tubule communicating with the first hollow chamber is connected. In the event this becomes necessary, replugging the connection tubules only requires comparatively minor surgical intervention.

Further advantages and details of the invention will be explained in the following in conjunction with the enclosed drawing, which also discloses further aims of the invention.

### BRIEF DESCRIPTION OF THE DRAWING

In the drawing depict:

FIG. 1 view of an embodiment example of a device according to the invention in the form of a gastric band,

FIG. 2 side view, partially in section, along line A-A of FIG. 1,

FIG. 3 cross section through the band along line B-B of FIG. 2,

FIG. 4 section through a segment of the device along line C-C of FIG. 1,

FIG. 5 a section through a segment of the device along line D-D of FIG. 1,

FIG. 6 perspective view of this embodiment example, and

FIG. 7 perspective view of an injection port for the connection to one of the connection tubule.

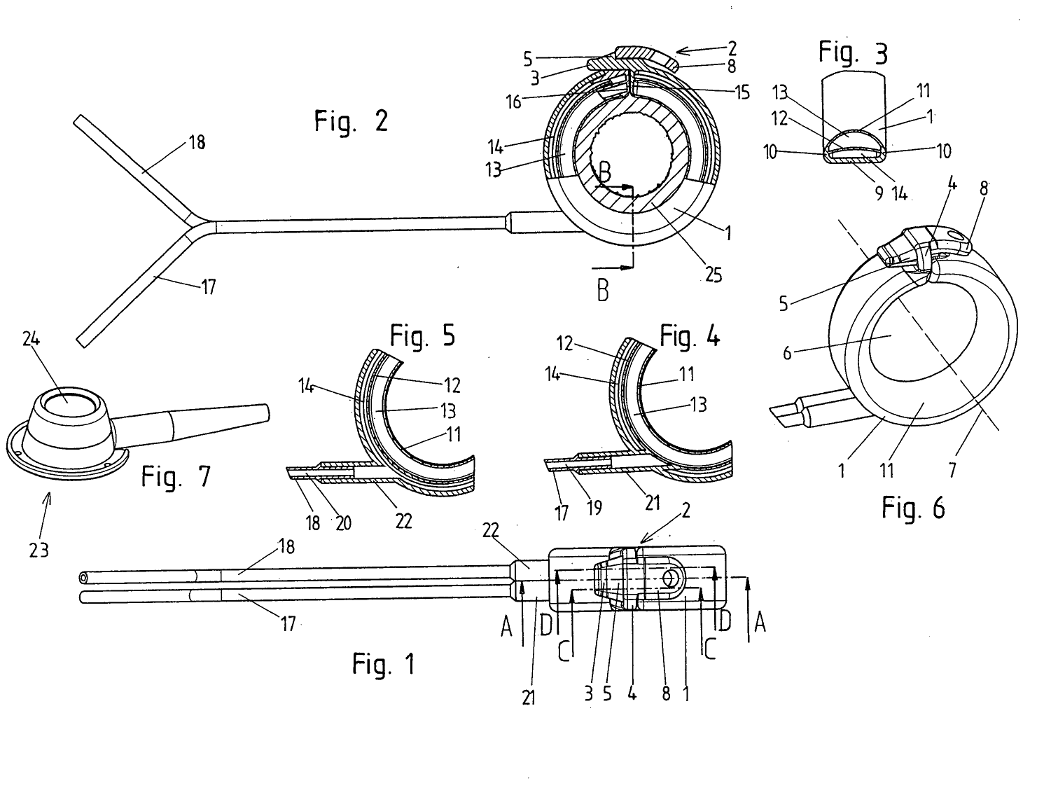

### DESCRIPTION OF THE PREFERRED EMBODIMENT EXAMPLES

The embodiment example depicted in the figures of a device according to the invention is developed as a gastric band and comprises a band 1 to be placed annularly about an appropriate portion 25 of the gastrointestinal tract, here the stomach, the gastric inlet or the esophagus indicated only schematically in FIG. 2 in cross section. The band is provided with parts of a closure device 2 in the proximity of its two ends. The closure device comprises at one end of the band 1 an extension 3 and a stay 4, disposed at the other end of the band, with an insertion opening for putting through the extension 3. To close the closure device 2, the extension 3 is pulled through the insertion opening in the stay 4, a catch lip 5 on the extension 3 becomes latched with the stay 4 and secures the closure device against unintentional opening.

In the closed state of the closure device 2 the band 1, which is now annular, encompasses a throughlet opening 6.

The catch lip 5 preferably projects beyond the side, facing away from the throughlet opening 6, and the edge sides of the extension 3, facing in the direction of an axis 7 of the throughlet opening 6, the catch lip 5 having a shape widening conically viewed from the free end of extension 3, in order to permit it to be pulled through the insertion opening of stay 4. On the side which in the inserted state extends behind the edge of the insertion opening of stay 4, the catch lip 5 is developed in the form of a step and projects approximately perpendicularly from extension 3.

The extension 3 with its catch lip 5 and the stay 4 are preferably comprised of an elastic material, in particular silicone, with a hardness and, further, with a cooperating geometric shape such that the closure device opens under a force-open tension exceeding a specified limit value. Thereby a safety closure can be provided, through which an impermissibly high pressure in the encompassed body organ can be avoided.

From stay 4 projects a pull tab 8 in the direction approximately opposite to the direction of insertion of extension 3, whereby closing the closure device 2 is facilitated by applying medical instruments on the pull tab, on the one hand, and on the other hand, on the extension 3 guided with its front end through the insertion opening in stay 4.

The parts of the closure device 2, disposed at both ends of the band 1, can be disposed on stoppers, which project into the ends of the band, are adhered in these ends and close these ends. The integral development from a single piece of material of the closure device, or parts thereof, with the band 1, in particular on the side of the extension 3, is conceivable and possible.

On the side, facing away from its throughlet opening 6, the band 1 comprises a back segment 9. On its two edge sides it is provided with prolongations 10 (which are significantly shorter than the width of the back segment 9) projecting in the direction toward the throughlet opening 6. On these prolongations are disposed two elastic diaphragms 11, 12 (=thin skins with delimiting function), which bridge the prolongations 10. The diaphragms 11, 12 can, for example, be adhered on the prolongations 10. The integral development from a single piece of material of one or both diaphragms 11, 12 with the prolongations 10 and the back segment 9 are conceivable and possible. For this purpose appropriate cores can be disposed in an injection molding form on the one hand, between the two diaphragms 11, 12 and, on the other hand, between the diaphragm 12 and the back segment 9, which, after the injection and the unmolding of the band, are removed from it. Furthermore, the diaphragms 11 and 12 could also be formed by two tubes one slid into the other, which, on the side facing away from the throughlet opening 6, are adhered with one another and with the back segment 9 and the prolongations 10.

The prolongations 10 can in principle also be omitted.

Between the diaphragm 11 adjoining the throughlet opening 6 and the diaphragm 12 a first hollow chamber 13 is formed, which extends at least over a large portion of the length of the band 1. Between diaphragm 12 and back segment 9 is formed a second hollow chamber 14, which also extends at least over a large portion of the length of band 1 and which is disposed radially outside the first hollow chamber 13. The embodiment example depicted in the figures shows the preferred implementation of the hollow chambers 13, 14 over the entire length of the band, the hollow chambers 13, 14 being closed at both ends of the band by terminal walls 15, 16.

Like the back segment 9 and the optionally provided prolongations 10, diaphragms 11, 12 are preferably comprised of silicone. It is preferred that the elastic extensibility of diaphragms 11, 12 is greater than that of the back segment 9. This can be attained for example by implementing the back segment 9 such that it has a greater thickness than the diaphragms 11, 12. Differing material hardnesses of the back segment 9 and of the diaphragms 11, 12 are also conceivable and possible. The back segment, further, could also be reinforced by a reinforcement layer, for example, embedded into the material of the back segment or adhered on the side facing away from the throughlet opening 6 and extending over the length of the band. This reinforcement layer can here have filaments continuous in the longitudinal direction and advantageously also in the transverse direction over its entire extent and can be developed in particular as a rectangular weave fabric, for example of a synthetic material.

By implementing the back segment as having low elasticity or no elasticity, its expansion when filling the band with a filling material is prevented.

The device comprises further two connection tubules 17, 18, whose inner channels 19, 20 communicate, on the one hand, with the first hollow chamber 13, and, on the other hand, with the second hollow chamber 14. For that purpose connecting pieces 21, 22 are disposed on band 1, for example implemented integrally with the band 1, which terminate, on the one hand, in the first hollow chamber 13, on the other hand, in the second hollow chamber 14, and in which the connection tubules 17, 18 are adhered with their end segments. The connecting pieces 21, 22 extend from the band 1 in a central region.

At each of the other ends of the connection tubules, for example, an injection port 23 can be connected, as is depicted in FIG. 7. The injection port 23 comprises in conventional manner a diaphragm 24, through which the needle of a syringe filled with the filling material, in particular sterile water, can be pierced through the skin of the patient in order to fill the corresponding hollow chamber 13, 14 of the band 1 with the desired quantity of filling material and thereby to adjust the opening cross section of the throughlet opening 6 in the desired manner. The relatively thick diaphragm 24 closes tightly again after the needle of the syringe has been withdrawn.

After the placement of the band, annularly closed by means of the closure device 2, about the body organ, first, the first hollow chamber 13 is filled with the desired quantity of filling material, such that the diaphragm 11 correspondingly expands in the direction toward axis 7 until the size of the throughlet opening 6, and consequently the size of the passageway opening of portion 25 of the gastrointestinal tract is adjusted to the desired value. The diaphragm 12 comes into contact on the back segment 9 of band 1.

If, due to a leak in the diaphragm 11, the hollow chamber becomes permeable, the filling material can escape from the hollow chamber 13, such that the diaphragm 11 loses its tension and the throughlet opening 6 becomes enlarged and the function of the band 1 is lost. In this case the second hollow chamber 14 can be filled with filling material, such that now the diaphragm 12 expands correspondingly in the direction toward axis 7 and moves diaphragm 11 along with it. Via the filling of hollow chamber 14 and the expansion of diaphragm 12, the size of the throughlet opening can consequently also be adjusted as desired even in the event a leak occurs in the diaphragm 11. The average length of use of the device is thereby overall significantly extended, such that the patient is spared from having to undergo surgical interventions.

Instead of an injection port depicted, a pumping device for filling the particular hollow chamber 13, 14 could be connected to the connection tubules 17 or 18. Such pumping device could include a reservoir with filling material, whose volume is variable. If the device is developed as an openable and closable closure, for example as an anal band, the reservoir would need to be switchable in known manner between two sizes of its volume corresponding to the closed and the open state of the device.

Different modifications of the described embodiment examples are conceivable and possible. The diaphragms 11, 12 could for example also be implemented as multilayer diaphragms. As the filling medium could also be provided other fluids, in particular liquids, which, for example, could have a higher viscosity than water.

### LEGEND TO THE REFERENCE NUMBERS

1 Band

2 Closure device

3 Extension

4 Stay

5 Catch lip

6 Throughlet opening

7 Axis

8 Pull tab

9 Back segment

10 Prolongation

11 Diaphragm

12 Diaphragm

13 Hollow chamber

14 Hollow chamber

15 Wall

16 Wall

17 Connection tubule

18 Connection tubule

19 Channel

20 Channel

21 Connecting piece

22 Connecting piece

23 Injection port

24 Diaphragm

25 Portion of the gastrointestinal tract

1. Device for generating an artificial constriction in the gastrointestinal tract with
a band (1), which can be placed annularly about a particular portion of the gastrointestinal tract, and
a closure device (2) to connect the end regions of the band (1) placed annularly about the portion of the gastrointestinal tract, which, in the closed state of the closure device (2), encompasses a throughlet opening (6),
the band (1) comprising a first hollow chamber (13) fillable with a filling medium extending at least over a large portion of the length of the band (1) and on its side facing the throughlet opening (6), delimited by a diaphragm (11), by means of which the size of the throughlet opening (6) can be reduced by filling the hollow chamber (13), and
the band further comprising a second hollow chamber (14), separated from this first hollow chamber (13) and fillable with a filling medium, which extends at least over a large portion of the length of the band (1) and which on its side, facing the throughlet opening (6), is delimited by a further diaphragm (12), by means of which the size of the throughlet opening (6) can be reduced by filling the second hollow chamber (14).

2. Device as claimed in claim 1, in which, in the annularly closed state of the band (1), the second hollow chamber (14) encompasses the first hollow chamber (13).

3. Device as claimed in claim 1, in which the diaphragm (12), delimiting the second hollow chamber (14) on its side facing the throughlet opening (6), delimits the first hollow chamber (13) on its side facing away from the throughlet opening (6).

4. Device as claimed in claim 1, in which the two diaphragms (11, 12) are elastically extensible and preferably comprised of silicone.

5. Device as claimed in claim 1, in which the band (1) on its side, facing away from the throughlet opening (6), comprises a back segment (9) less extensible than the diaphragms (11, 12), which is preferably formed at least partially of a silicone.

6. Device as claimed in claim 5, in which the diaphragms (11, 12) are disposed on the back segment (9) or on prolongations (10) projecting therefrom at its two edge sides in the direction toward the throughlet opening (6).

7. Device as claimed in claim 1, the band (1) comprising at its two ends walls (15, 16) closing the hollow chambers (13, 14).

8. Device as claimed in claim 1, in which the device comprises first and second connection tubules (17, 18) with inner channels (19, 20), the inner channel (19) of the one connection tubule (17) communicating with the first hollow chamber (13) and the inner channel (20) of the other connection tubule (18) communicating with the second hollow chamber (14).

9. Device as claimed in claim. 8, in which the connection tubules (17, 18) are disposed on the connecting piece (21, 22), of which the one connecting piece (21) terminates in the first hollow chamber (13) and the other connecting piece (22) in the second hollow chamber (14).
